(19) United States
(12) Patent Application Publication
(10) Pub. No.: US 2020/0398182 A1
(43) Pub. Date: Dec. 24, 2020

(54) SYSTEM AND METHOD FOR SUBSTANCE EXTRACTION

(71) Applicant: Aftermath Labs LLC, Los Angeles, CA (US)

(72) Inventors: William C. Ragsdale, Sparks, NV (US); Devon Miller, Los Angeles, CA (US); William C. Ragsdale, Sparks, NV (US); Devon Miller, Los Angeles, CA (US)

(21) Appl. No.: 16/902,202

(22) Filed: Jun. 15, 2020

Related U.S. Application Data
(60) Provisional application No. 62/861,251, filed on Jun. 13, 2019.

(51) Int. Cl. B01D 3/14; B01D 3/10; B01D 3/38; B01D 9/04; C07D 311/80

(52) U.S. Cl. CPC B01D 3/143; B01D 3/10; B01D 2009/009; B01D 9/04; C07D 311/80; B01D 3/38

(57) ABSTRACT

A system and method for separating desirable elements from typically undesirable byproducts of a cannabis extraction system is disclosed. The system and method may separate terpenes and/or THC from residue byproducts that may typically be discarded as waste. The system and method includes at least two unique steps of refinement that may result in purity levels of 90% for the terpenes and 90% for the THC.

### RELATIONSHIPS TO PRIOR APPLICATIONS

This application claims priority to U.S. Provisional Application No. 62/861,251, filed Jun. 13, 2019, the entire contents of which are hereby fully incorporated herein by reference for all purposes.

### COPYRIGHT STATEMENT

This patent document contains material subject to copyright protection. The copyright owner has no objection to the reproduction of this patent document or any related materials in the files of the United States Patent and Trademark Office, but otherwise reserves all copyrights whatsoever.

### FIELD OF THE INVENTION

This invention relates to the extraction process of substances, including the extraction of cannabinoids, terpenes, and other volatile substances, from cannabis plant materials.

### BACKGROUND

With the legalization of many products produced from cannabis plants in many states, a variety of systems and methods has been developed to separate, extract, and refine desirable substances from the raw cannabis plant materials.

However, many of these systems and methods produce high amounts of byproducts that are considered undesirable and that are discarded and/or disposed of as waste. The ratio of the desirable outputs (quality and composition of the extract; e.g. the purity of cannabinoids or the ratio of cannabinoids to other volatile elements) to the total plant material input (commonly referred to as “biomass”) is an efficiency metric which can be used to quantify a given process. Additionally, the purity of the extract (the ratio of desirable vs. undesirable outputs) can be used to further investigate the efficacy of a given process. This is achieved by comparing the given amount of desirable materials in the initial and post-treatment biomass to the amount and purity of the outputs. Given the significant amounts of waste products produced by current systems and methods for substance(s) separations, these systems and methods may provide a sub-optimal efficiency factor, but because the yield of desirable elements using these systems and methods may be profitable, the undesirable byproducts secondarily removed are discarded without consideration.

Accordingly, there is a need for extraction/separation systems and methods that may decrease the amounts of undesirable byproducts. There is also a need for such systems and methods that may refine byproducts, which are typically viewed as undesirable, into products that may be considered as desirable.

### BRIEF DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the several views, and wherein:

FIG. 1 shows aspects of an example distillation system according to exemplary embodiments hereof; and

FIG. 2 shows a generalized diagram of the process methodology.

### DETAILED DESCRIPTION OF THE EXEMPLARY EMBODIMENTS

The following detailed description is not intended to limit the current invention. Alternate embodiments and variations of the subject matter described herein will be apparent to those of ordinary skill in the art.

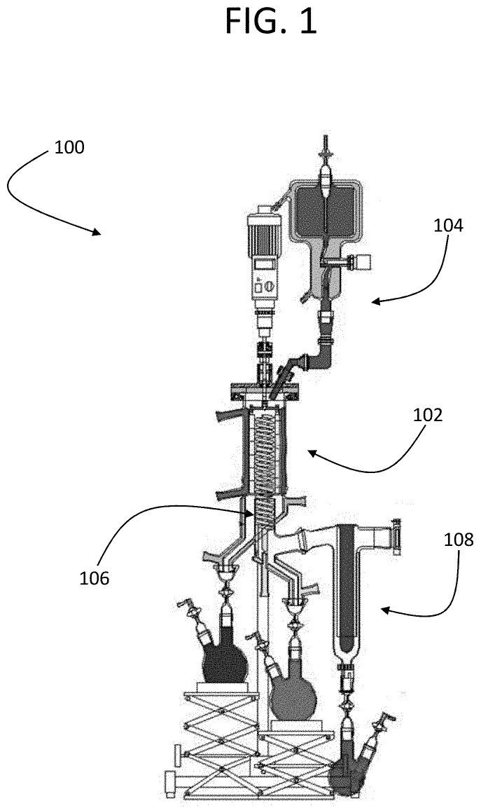

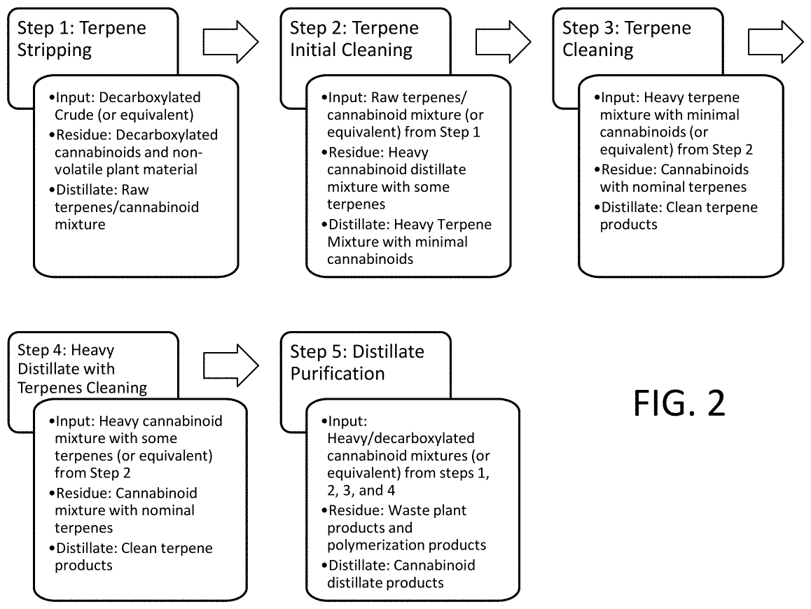

Systems and methods according to exemplary embodiments hereof are described with reference to FIGS. 1-2. Where the same or similar components appear in more than one figure, they are identified by the same or similar reference numerals.

In general, the invention according to exemplary embodiments hereof provides a system and method that may separate one or more components or substances from one or more materials, mixtures or compounds. In one exemplary embodiment hereof, the system and method may extract and/or distill one or more components or substances from one or more plant materials. In one exemplary embodiment, the system and method may extract and/or distill cannabinoids (which includes, but are not limited to, natural isomers of tetrahydrocannabinol, tetrahydrocannabinolic acid, cannabinol, cannabinolic acid, cannabidiol, cannabidiolic acid, cannabigerol, cannabigerolic acid, etc.), terpenes and sesquiterpenes, carbohydrates, proteins, fats, oils, minerals, and any combination thereof from cannabis plant materials.

In one exemplary embodiment hereof, the system and method may extract and/or distill cannabinoids (which includes, but are not limited to natural isomers of tetrahydrocannabinol, tetrahydrocannabinolic acid, cannabinol, cannabinolic acid, cannabidiol, cannabidiolic acid, cannabigerol, cannabigerolic acid, etc.) and the terpenes and sesquiterpenes from cannabis plant materials with a resultant higher purity output composition compared to other known methods of extraction and purification.

One process of separating a particular substance from plant material may include steps of extraction followed by steps of refinement or distillation. The extraction process also may include a process referred to as winterization.

As is known in the art, one process of extraction may involve using a solvent to isolate one or more of the desired components (and/or waste, substances) from a crude solid or liquid mixture. In some embodiments, it may be preferable that the solvent be immiscible with the crude mixture, and that the solvent effectively dissolve the desired component(s) being isolated. The desired component(s) may then transfer from the solid or liquid mixture to the solvent. The component(s) may then be separated from the solvent using other methods.

The extraction process commonly known as winterization (also referred to as fractionate crystallization) may involve dissolving the untreated extract in a solvent (e.g., methanol, ethanol, propanol, hexanes, petroleum ether, ether, pentane, heptane, dimethylsulfoxide, etc.) and then reducing the temperature of the solution sufficiently such that undesirable chlorophylls and other pigments, long carbon chain fatty acids, oil, and/or waxes precipitate. This temperature may be between 0° C. to −20° C., −20° C. to −40° C., −40° C. to −60° C., −60° C. to −80° C. The precipitates may subsequently be removed using other methods such as filtration, centrifugation, decantation, other separation processes and any combination thereof. Upon solvent removal, the oil remaining may include desirable components such as cannabinoids and other components, that may be further refined and processed for consumer use if desired.

However, it should be noted that winterization may also remove some desirable substances from the mixture such as terpenes and cannabinoids, representing a step where efficiency can be lost. That is, the desirable components may be removed along with the fats, waxes and plant chlorophyll, and these byproducts together may be therefore discarded as waste. When terpenes are lost at a higher rate than the other desirable components, terpenes may be reintroduced into the final product during the later stages of the production process to achieve a more desirable product.

Example extraction techniques may include (without limitation) maceration, infusion, percolation, digestion, decoction, hot continuous extraction (Soxhlet), aqueous-alcoholic extraction by fermentation, liquid-liquid extraction, counter-current liquid-liquid extraction, microwave-assisted extraction, ultrasound extraction (sonication), supercritical fluid extraction (e.g., supercritical CO2, butane, or propane extraction), winterization and other types of extraction methods.

The extraction process may then sometimes be followed by a refinement process, generally either chromatography or distillation. As is also known in the art, a process of distillation may involve the action of separating one or more components or substances from a crude mixture using variances in boiling and condensation points of the given components. The technique may exploit the differences between the boiling points of the different components within the crude mixture. As is known, more volatile substances may boil at lower temperatures while less-volatile substances may boil at higher temperatures. In one example, to separate the first component from a crude mixture, the temperature of the crude mixture may be increased to the boiling point of only the first desired component. Once the specific boiling point of the desired component is reached, the substance will generally boil into the vapor phase and subsequently condense on a surface that is less than the boiling temperature of the component to provide a distillate. The temperature of the mixture may then be further increased, resulting in the substance with the next lowest boiling point being vaporized from the crude mixture and condensed on a surface to provide the refined compound. In some embodiments, the materials may be collected together to provide a mixture of desirable components or collected in fractions to provide purified compounds. The process may be continued until all the desired substances have been collected.

Example distillation techniques may include (without limitation) simple distillation, fractional distillation, steam distillation, vacuum distillation, air-sensitive vacuum distillation, short path distillation, zone distillation, and other types of distillation.

As is known, typical extraction and/or distillation processes used to extract desired substances (e.g., cannabinoids and/or terpenes) from plant materials (e.g., cannabis plant materials or “biomass”) also may output a high percentage of byproducts that may not be considered as desirable. Because of this, the byproducts are typically discarded as waste materials. The ratio of the desirable outputs (cannabinoids and/or terpenes in this example) to the total plant material input (the cannabis plant material) is an efficiency metric used to quantify the process. In this way, the efficiency metric reflects a measurement of the resulting amount of desirable outputs vs. the resulting amount of undesirable outputs.

Depending on the plant material and the types of extraction/distillation processes employed, the byproducts may include not only undesirable substances like fats, waxes, lipids and plant chlorophyll, but also highly desirable substances such as terpenes and cannabinoids. This represents an area where efficiency would lead to a higher return on a given plant material. However, because the desirable substances (e.g., terpenes and cannabinoids) may be combined with the undesirable substances to form a single solution of byproducts, when the byproducts are discarded the terpenes and cannabinoids contained therein are also discarded.

In one exemplary embodiment hereof, the system and method according to the present invention may convert byproduct outputs, traditionally considered to be waste products, into desirable outputs, hence improving the efficiency metric. In general, the process may begin with extraction and/or distillation processes that may output both desirable substances and byproducts. The desirable substances may be subsequently refined and processed for use in consumer products. However, instead of discarding the byproducts as waste material, the chemical compositions of the byproducts are analyzed and then further processed according to the present invention to recover the otherwise lost desired elements of the mixture.

For the purposes of this specification, but not to be seen as a limitation on extraction solvent or distillation processes, the systems and methods will be described with relation to an extraction and/or distillation process that uses ethanol as a solvent to extract both desirable and undesirable substances from the plant material, and short path fractional distillation to further process, refine and/or purify the substances. A general schematic of a short path fractional distillation system 100 is shown in FIG. 1 and a general process flow diagram is shown in FIG. 2. This system may also include a step for removing solvent such as wiped film evaporation, falling film evaporation, rising film evaporation, or spray drying.

However, it is understood by a person of skill in the art, upon reading this specification, that the systems and methods according to the current invention may be applied and utilized with any type of appropriate extraction and/or distillation techniques and/or systems, and that the scope of the system and method is not limited in any way by the type of extraction and/or distillation techniques and/or hardware/software systems used.

As shown in FIG. 1, the distillation system 100 may include an evaporator 102, a feed 104, a condenser 106 and a cold trap 108. The system 100 also may include other elements and components necessary to fulfill its functionalities. When in use, the evaporator 102 may be set to an evaporator temperature, the feed 104 may be set to a feed temperature, the condenser 106 may be set to a condenser temperature and the cold trap 108 may be set to a cold trap temperature. The pressure may be set at a controlled vacuum level. For refinement of the byproduct material, the temperature settings must be specifically designed according to the chemical compositions within the byproducts. In this way, the desirable elements (e.g., the lost terpenes) may be separated from the undesirable elements and removed from the overall mixture of collected byproducts.

In one exemplary embodiment hereof, the process includes the separation of cannabinoids and other desired substances from cannabis plant material by suspending the cannabinoids and desired substances in a solvent (e.g., methanol, ethanol, propanol, acetone, toluene, benzene, dichloromethane, chloroform, ether, petroleum ether, hexanes, heptane), and subsequently performing additional processing steps to isolate the desirable components into a highly purified form and solvent removal. The process also may induce decarboxylation of any applicable desirable outputs (e.g., cannabigerolic acid to cannabigerol, cannabidiolic acid to cannabidiol, tetrahydrocannabinolic acid to tetrahydrocannabinol), and the resultant material may be referred to as a decarboxylated distillate or crude (depending on if decarboxylation is performed prior to extraction).

The decarboxylated crude may be further processed using a method of distillation (e.g., using the distillation system 100 of FIG. 1 or any other distillation or refinement system). The initial steps of the distillation/refinement process (e.g., Steps 1-3) may output desired substances as well as byproducts (typically considered waste and discarded as such) as described above.

The byproducts and desirable components may include some or all of the following substances and/or elements (without limitation):
1. Chlorophyll and other plant pigments;
2. Extraction solvents (e.g., methanol, ethanol, propanol, acetone, toluene, benzene, dichloromethane, chloroform, ether, petroleum ether, hexanes);
3. Agricultural byproducts (e.g., pesticides, nitrates, dirt, minerals, etc.);
4. Industrial byproducts (heavy metals);
5. Water;
6. Plant matter such as fats, lipids, waxes, plant proteins, plant hormones, cellulose, fiber, carbohydrates, sugars, and other types of plant matter;
7. Terpenes and sesquiterpenes such as (without limitation) Limonene, Pinene, Linalool, Caryophyllene, Humulene, any isomers of the above listed and other naturally derived terpenes; and
8. Natural cannabinoids such as Tetrahydrocannabinol (THC), Cannabigerol (CBG), Cannabinol (CBN), Cannabichromene (CBC), Cannabicyclol (CBL), Cannabitriol (CBT), Cannabicitran (CRM), Cannabifuran (CBF) and others. Note that at least 113 different cannabinoids are known to occur in the cannabis plant and that the described byproducts may include any and/or all the known cannabinoids as well as those yet to be discovered. In some embodiments, the concentration of the cannabinoids within the crude may be about 30% to 90%.

The ensuing steps of the exemplary method (e.g., Steps 4-5) may then further process the byproducts mixtures in order to recover lost substances that when properly processed may be desirable. As shown in FIG. 2, these steps may include at least the following steps (without limitation):

Step 1: Terpene Stripping Step

The input to the distillation system 100 for Step 1 may include at least some of the following (without limitation): Decarboxylated crude.

It may be preferable that at least some of the input decarboxylated crude include some of the output from the ethanol extraction process described above, but it is understood that the decarboxylated crude may come from a different process(es) or from any combination thereof.

The temperature range settings are dependent on the composition of the components and desired outputs, therefore the range of settings of the distillation system 100 elements for Step 1 may include:
Evaporator 102: Preferably 150° C. to 160° C. Other suitable temperature ranges include: 100° C. to 110° C.; 110° C. to 120° C.; 120° C. to 130° C.; 130° C. to 140° C.; 150° C. to 160° C.; 160° C. to 170° C.; 170° C. to 180° C.; 180° C. to 190° C.; 190° C. to 200° C.; and 210° C. to 220° C.;
Feed 104: Preferably 100° C. to 110° C. Other suitable temperature ranges include: 70° C. to 80° C.; 80° C. to 90° C.; 90° C. to 100° C.; 100° C. to 110° C.; 110° C. to 120° C.; 120° C. to 130° C.; 130° C. to 140° C.; and 140° C. to 150° C.;
Condenser 106: Preferably 15° C. to 40° C. Other suitable temperature ranges include: −20° C. to −10° C.; −10° C. to 0° C.; 0° C. to 10° C.; 10° C. to 20° C.; 20° C. to 30° C.; 30° C. to 40° C.; 40° C. to 50° C.; and 60° C. to 70° C.;
Cold Trap 108: Preferably −40° C. to −50° C. Other suitable temperature ranges include: −100° C. to −90° C.; −90° C. to −80° C.; −80° C. to −70° C.; −70° C. to −60° C.; −60° C. to −50° C.; −50° C. to −40° C.; and −40° C. to −30 ° C.

Upon processing the decarboxylated crude within these settings, the outputs of the distillation system 100 for Step 1 may include at least some of the following (without limitation):
a. Residue: Decarboxylated Cannabinoids and Terpene Stripped Crude
b. Distillate: Raw Terpene and Distillate Mixture
c. Cold Trap: Lighter terpenes and residual extraction solvent

Step 2: Terpene Initial Cleaning Run

The input to the distillation system 100 for Step 2 may include at least some of the following (without limitation): Raw terpene and distillate mixture. Note; similar mixtures can be used regardless of production method.

It may be preferable that at least some of the input raw terpene and distillate mixture for this step include at least some of the output (b) from Step 1 above, but it is understood that input raw terpene and distillate mixture may come from a different process(es) or from any combination thereof.

The temperature settings are dependent on the desired material separations, therefore the settings of the distillation system 100 elements for Step 2 may include:
Evaporator 102: Preferably 145° C. or from 140° C. to 150° C. Other suitable temperature ranges include: 110° C. to 120° C.; 120° C. to 130° C.; 130° C. to 140° C.; 140° C. to 150° C.; 150° C. to 160° C.; 160° C. to 170° C.; 170° C. to 180° C.;
Feed 104: Preferably 110° C. or from 105° C. to 115° C. Oher suitable temperature ranges include: 80° C. to 90° C.; 90° C. to 100° C.; 110° C. to 120° C.;
Condenser 106: Preferably 25° C. or from 20° C. to 30° C. Other suitable temperature ranges include: 5° C. to 15° C.; 15° C. to 25° C.; 25° C. to 35° C.;
Cold Trap 108: Preferably −40° C. to −50° C. Other suitable temperature ranges include: −100° C. to −90° C.; −90° C. to −80° C.; −80° C. to −70° C.; −70° C. to −60° C.; −6 ° C. to −50° C.; −50° C. to −40° C.; −40° C. to −30° C.; −30° C. to −20° C.

Upon processing the raw terpene and distillate mixture at these settings, the outputs of the distillation system 100 for Step 2 may include at least some of the following (without limitation):
a. Residue: Heavy Distillate mixture with Terpenes;
b. Distillate: Heavy terpene mixture with minimal distillate;
c. Cold Trap: Nil.

Step 3: Terpene Cleaning Run #2

The input to the distillation system 100 for Step 3 may include at least some of the following (without limitation): Heavy terpene mixture with minimal distillate.

It may be preferable that at least some of the input heavy terpene mixture with minimal distillate for this step include at least some of the output (b) from Step 2 above, but it is understood that input heavy terpene mixture with minimal distillate may come from a different process(es) or from any combination thereof.

The temperature settings are dependent on the desired material separations, therefore the settings of the distillation system 100 elements for Step 3 may include:
Evaporator 102: Preferably 145° C. or from 140° C. to 150° C. Other suitable temperature ranges include: 110° C. to 120° C.; 120° C. to 130° C.; 130° C. to 140° C.; 140° C. to 150° C.; 150° C. to 160° C.; 160° C. to 170° C.; 170° C. to 180° C.;
Feed 104: Preferably 110° C. or from 105° C. to 115° C. Other suitable temperature ranges include: 80° C. to 90° C.; 90° C. to 100° C.; 110° C. to 120° C.;
Condenser 106: Preferably 25° C. or from 20° C. to 30° C. Other suitable temperature ranges include: 5° C. to 15° C.; 15° C. to 25° C.; 25° C. to 35° C.;
Cold Trap 108: Preferably −40° C. to −50° C. Other suitable temperature ranges include: −100° C. to −90° C.; −90° C. to −80° C.; −80° C. to −70° C.; −70° C. to −60° C.; −60° C. to −50° C.; −50° C. to −40° C.; −40° C. to −30° C.; −30° C. to −20° C.

Upon processing the heavy terpene mixture with minimal distillate at these settings, the outputs of the distillation system 100 for Step 3 may include at least some of the following (without limitation):
a. Residue: Heavy Distillate mixture with Terpenes
b. Distillate: Clean terpene product (final product)
c. Cold Trap: Nil

Note that the output in (b) above (the clean terpene product) may be a desired element and may be used as such in consumer products or otherwise. Also note that the residue output in (a) (the Heavy Distillate mixture with Terpenes) is typically considered an undesirable byproduct and is normally discarded as such. However, according to exemplary embodiments of the current invention, this byproduct may be further refined to produce desirable elements (e.g., by following Steps 4-5 below).

Step 4: Heavy Distillate Mix With Terpene Cleaning Run

The input to the distillation system 100 for Step 4 may include at least some of the following (without limitation): Heavy distillate mixture with terpenes.

It may be preferable that at least some of the input heavy distillate mixture with terpenes for this step include at least some of the output (a) from Step 3 above, but it is understood that input heavy distillate mixture with terpenes may come from a different process(es) or from any combination thereof. Note that this residue may typically be discarded as waste but may instead be further refined in this step according to exemplary embodiments of the current invention hereof.

The temperature settings are dependent on the desired material separations, therefore the settings of the distillation system 100 elements for Step 4 may include:
Evaporator 102: Preferably 148° C. to 151° C. Other suitable temperature ranges include: 130° C. to 140° C.; 140° C. to 150° C.; 150° C. to 160° C.; 160° C. to 170° C.; 170° C. to 180° C.;
Feed 104: Preferably 110° C. or from 105° C. to 115° C. Other suitable temperature ranges include: 90° C. to 100° C.; 110° C. to 120° C.;
Condenser 106: Preferably 25° C. or from 20° C. to 30° C. Other suitable temperature ranges include: 5° C. to 15° C.; 15° C. to 25° C.; 25° C. to 35° C.;
Cold Trap 108: Preferably −40° C. to −50° C. Other suitable temperature ranges include: −60° C. to −50° C.; −50° C. to −40° C.; −40° C. to -−° C.; −30° C. to −20° C.

Upon processing the heavy distillate mixture with terpenes at these settings, the outputs of the distillation system 100 for Step 4 may include at least some of the following (without limitation):
Residue: Heavy distillate mixture with a nominal amount of terpenes
Distillate: Clean terpene products (this may include a final product)
Cold Trap: Nil

Note that the output in (b) above (the clean terpene products) may be a desired element and may be used as such in consumer products or otherwise. In some exemplary embodiments hereof, the purity level of the resulting terpene distillate may be at least 80%. In some exemplary embodiments herein, the purity level of the resulting terpene distillate may be 90% or greater.

In some embodiments, the concentration of terpenes in the resulting residue from this step is about 10% to 50%.

Also note that the residue output in (a) (the Heavy Distillate mixture with a nominal amount of terpenes) is typically considered an undesirable byproduct and is normally discarded as such. However, according to exemplary embodiments of the current invention, this byproduct may be further refined to produce desirable elements (e.g., by following Step 5 below).

Step 5: Distillate Run

The input to the distillation system 100 may include: Heavy distillate mixture with a nominal amount of terpenes.

It may be preferable that at least some of the input heavy distillate mixture with a nominal amount of terpenes for this step include at least some of the output (a) from Step 4 above, but it is understood that input distillate mixture with (or without) a nominal amount of terpenes may come from a different process(es) or from any combination thereof. Note that this residue may typically be discarded as waste but may instead be further refined in this step according to exemplary embodiments of the current invention hereof.

The temperature settings are dependent on the desired material separations, therefore the settings of the distillation system 100 elements for Step 5 may include:
Evaporator 102: Preferably 180° C. or from 175° C. to 185° C. Other suitable temperature ranges include: 150° C. to 160° C.; 160° C. to 170° C.; 170° C. to 180° C.; 180° C. to 190° C.; 190° C. to 200° C.; 200° C. to 210° C.;
Feed 104: Preferably 110° C. or from 105° C. to 115° C. Other suitable temperature ranges include: 90° C. to 100° C.; 110° C. to 120° C.;
Condenser 106: Preferably 110° C. or from 105° C. to 115° C. Other suitable temperature ranges include: 80° C. to 90° C.; 90° C. to 100° C.; 110° C. to 120° C.; 120° C. to 130° C.;
Cold Trap 108: Preferably −40° C. to −50° C. Other suitable temperature ranges include: −60° C. to −50° C.; −50° C. to −40° C.; −40° C. to −30° C.; −30° C. to −20° C.

Upon processing the heavy distillate mixture with a nominal amount of terpenes at these settings, the outputs of the distillation system 100 for Step 5 may include at least some of the following (without limitation):
Residue: Waste plant product (commonly referred to as distillation bottoms, but may be referred to as magma);
Distillate: cannabinoid distillate, high purity (this may include a final product);
Cold Trap: Nil.

Note that the output in (b) above (the cannabinoid distillate) may be a desired element and may be used as such in consumer products or otherwise. In some exemplary embodiments hereof, the purity level of the resulting cannabinoid distillate may be at least 80% or at least 90%. In some exemplary embodiments hereof, the purity level of the resulting THC distillate may be at least 80%, or at least 90%.

In some embodiments, the concentration of terpenes in the resulting residue from this step is about 1% to 20%.

A person of ordinary skill in the art will understand, that any method described above or below and/or claimed and described as a sequence of steps is not restrictive in the sense of the order of steps.

Those of ordinary skill in the art will appreciate and understand, upon reading this description, that embodiments hereof may provide different and/or other advantages, and that not all embodiments or implementations need have all advantages.

Where a process is described herein, those of ordinary skill in the art will appreciate that the process may operate without any user intervention. In another embodiment, the process includes some human intervention (e.g., a step is performed by or with the assistance of a human).

As used herein, including in the claims, the phrase “at least some” means “one or more,” and includes the case of only one. Thus, e.g., the phrase “at least some ABCs” means “one or more ABCs”, and includes the case of only one ABC.

As used herein, including in the claims, term “at least one” should be understood as meaning “one or more”, and therefore includes both embodiments that include one or multiple components. Furthermore, dependent claims that refer to independent claims that describe features with “at least one” have the same meaning, both when the feature is referred to as “the” and “the at least one”.

As used in this description, the term “portion” means some or all. So, for example, “A portion of X” may include some of “X” or all of “X”. In the context of a conversation, the term “portion” means some or all of the conversation.

As used herein, including in the claims, the phrase “using” means “using at least,” and is not exclusive. Thus, e.g., the phrase “using X” means “using at least X.” Unless specifically stated by use of the word “only”, the phrase “using X” does not mean “using only X.”

As used herein, including in the claims, the phrase “based on” means “based in part on” or “based, at least in part, on,” and is not exclusive. Thus, e.g., the phrase “based on factor X” means “based in part on factor X” or “based, at least in part, on factor X.” Unless specifically stated by use of the word “only”, the phrase “based on X” does not mean “based only on X.”

In general, as used herein, including in the claims, unless the word “only” is specifically used in a phrase, it should not be read into that phrase.

As used herein, including in the claims, the phrase “distinct” means “at least partially distinct.” Unless specifically stated, distinct does not mean fully distinct. Thus, e.g., the phrase, “X is distinct from Y” means that “X is at least partially distinct from Y,” and does not mean that “X is fully distinct from Y.” Thus, as used herein, including in the claims, the phrase “X is distinct from Y” means that X differs from Y in at least some way.

It should be appreciated that the words “first,” “second,” and so on, in the description and claims, are used to distinguish or identify, and not to show a serial or numerical limitation. Similarly, letter labels (e.g., “(A)”, “(B)”, “(C)”, and so on, or “(a)”, “(b)”, and so on) and/or numbers (e.g., “(i)”, “(ii)”, and so on) are used to assist in readability and to help distinguish and/or identify, and are not intended to be otherwise limiting or to impose or imply any serial or numerical limitations or orderings. Similarly, words such as “particular,” “specific,” “certain,” and “given,” in the description and claims, if used, are to distinguish or identify, and are not intended to be otherwise limiting.

As used herein, including in the claims, the terms “multiple” and “plurality” mean “two or more,” and include the case of “two.” Thus, e.g., the phrase “multiple ABCs,” means “two or more ABCs,” and includes “two ABCs.” Similarly, e.g., the phrase “multiple PQRs,” means “two or more PQRs,” and includes “two PQRs.”

The present invention also covers the exact terms, features, values and ranges, etc. in case these terms, features, values and ranges etc. are used in conjunction with terms such as about, around, generally, substantially, essentially, at least etc. (i.e., “about 3” or “approximately 3” shall also cover exactly 3 or “substantially constant” shall also cover exactly constant).

As used herein, including in the claims, singular forms of terms are to be construed as also including the plural form and vice versa, unless the context indicates otherwise. Thus, it should be noted that as used herein, the singular forms “a,” “an,” and “the” include plural references unless the context clearly dictates otherwise.

Throughout the description and claims, the terms “comprise”, “including”, “having”, and “contain” and their variations should be understood as meaning “including but not limited to”, and are not intended to exclude other components unless specifically so stated.

It will be appreciated that variations to the embodiments of the invention can be made while still falling within the scope of the invention. Alternative features serving the same, equivalent or similar purpose can replace features disclosed in the specification, unless stated otherwise. Thus, unless stated otherwise, each feature disclosed represents one example of a generic series of equivalent or similar features.

The present invention also covers the exact terms, features, values and ranges, etc. in case these terms, features, values and ranges etc. are used in conjunction with terms such as about, around, generally, substantially, essentially, at least etc. (i.e., “about 3” shall also cover exactly 3 or “substantially constant” shall also cover exactly constant).

Use of exemplary language, such as “for instance”, “such as”, “for example” (“e.g.,”) and the like, is merely intended to better illustrate the invention and does not indicate a limitation on the scope of the invention unless specifically so claimed.

While the invention has been described in connection with what is presently considered to be the most practical and preferred embodiments, it is to be understood that the invention is not to be limited to the disclosed embodiment, but on the contrary, is intended to cover various modifications and equivalent arrangements included within the spirit and scope of the appended claims.

What is claimed is:

1. A method of separating terpenes and tetrahydrocannabinol (THC) from distillation byproducts, the method comprising:
(A) obtaining a decarboxylated crude from an extraction process;
(B) using a distillation system to distill the decarboxylated crude obtained in (A) into a first distillate comprising a raw terpene and distillate mixture;
(C) using the distillation system to distill the first distillate obtained in (B) into a second distillate comprising a heavy terpene and distillate mixture;
(D) using the distillation system to distill the second distillate obtained in (C) into a first residue comprising a first heavy distillate mixture with a first concentration of terpenes;
(E) using the distillation system to separate the first residue obtained in (D) into separated terpenes and a second residue comprising a second heavy distillate mixture with a second concentration of terpenes, the second concentration of terpenes being less than the first concentration of terpenes;
(F) using the distillation system to distill the second residue obtained in (E) into a THC distillate;
wherein the separated terpenes in (E) includes a first terpene purity level and the TCH distillate in (F) includes a first THC purity level.

2. The method of claim 1 wherein the first terpene purity level is at least 80% and/or the first THC purity level is at least 80%.

3. The method of claim 1 wherein the distillation system includes an evaporator set to an evaporator temperature, a feed set to a feed temperature, a condenser set to a condenser temperature, and a cold trap set to a cold trap temperature, wherein the evaporator temperature in (D) is about 130° C. to 180° C., the feed temperature in (D) is about 90° C. to 120° C., the condenser temperature in (D) is about 5° C. to 35° C., and the cold trap temperature in (D) is about −60° to −20° C.

4. The method of claim 1 wherein the distillation system includes an evaporator set to an evaporator temperature, a feed set to a feed temperature, a condenser set to a condenser temperature, and a cold trap set to a cold trap temperature, wherein the evaporator temperature in (E) is about 150° C. to 210° C., the feed temperature in (E) is about 90° C. to 120° C., the condenser temperature in (E) is about 80° C. to 130° C., and the cold trap temperature in (E) is about −60° to −20° C.

5. The method of claim 1 wherein the distillation system includes an evaporator set to an evaporator temperature, a feed set to a feed temperature, a condenser set to a condenser temperature, and a cold trap set to a cold trap temperature, wherein the evaporator temperature in (D) is about 145° C. to 155° C., the feed temperature in (D) is about 105° C. to 115° C., the condenser temperature in (D) is about 20° C. to 30° C., and the cold trap temperature in (D) is about −50° to −40° C.

6. The method of claim 1 wherein the distillation system includes an evaporator set to an evaporator temperature, a feed set to a feed temperature, a condenser set to a condenser temperature, and a cold trap set to a cold trap temperature, wherein the evaporator temperature in (E) is about 175° C. to 185° C., the feed temperature in (E) is about 105° C. to 115° C., the condenser temperature in (E) is about 105° C. to 115° C., and the cold trap temperature in (E) is about −50° to −40° C.

7. The method of claim 1 wherein the first concentration of terpenes in (D) is about 10% to 50%.

8. The method of claim 1 wherein the second concentration of terpenes in (E) is about 1% to 20%.

9. The method of claim 1 wherein the cannabinoid concentration of the decarboxylated crude obtained in (A) is about 50% to 85%.

10. A method of separating terpenes and tetrahydrocannabinol (THC) from distillation byproducts, the method comprising:
(A) obtaining a first distillation byproduct, the first distillation byproduct a result of distilling a decarboxylated crude three times, and comprising a first residue comprising a first heavy distillate mixture with a first concentration of terpenes;
(B) using a distillation system to separate the first distillation byproduct obtained in (A) into separated terpenes and a second distillation byproduct comprising a second residue of a second heavy distillate mixture with a second concentration of terpenes, the second concentration of terpenes being less than the first concentration of terpenes;
(C) using a distillation system to separate the second distillation byproduct obtained in (B) into a THC distillate and a third distillation byproduct;
wherein the separated terpenes in (B) include a first terpene purity level and the TCH distillate in (C) includes a first THC purity level.

11. The method of claim 10 wherein the first terpene purity level is at least 80% and/or the first THC purity level is at least 80%.

12. The method of claim 10 wherein the distillation system includes an evaporator set to an evaporator temperature, a feed set to a feed temperature, a condenser set to a condenser temperature, and a cold trap set to a cold trap temperature, wherein the evaporator temperature in (D) is about 130° C. to 180° C., the feed temperature in (D) is about 90° C. to 120° C., the condenser temperature in (D) is about 5° C. to 35° C., and the cold trap temperature in (D) is about −60° to −20° C.

13. The method of claim 10 wherein the distillation system includes an evaporator set to an evaporator temperature, a feed set to a feed temperature, a condenser set to a condenser temperature, and a cold trap set to a cold trap temperature, wherein the evaporator temperature in (E) is about 150° C. to 210° C., the feed temperature in (E) is about 90° C. to 120° C., the condenser temperature in (E) is about 80° C. to 130° C., and the cold trap temperature in (E) is about −60° to −20° C.

14. The method of claim 10 wherein the distillation system includes an evaporator set to an evaporator temperature, a feed set to a feed temperature, a condenser set to a condenser temperature, and a cold trap set to a cold trap temperature, wherein the evaporator temperature in (D) is about 145° C. to 155° C., the feed temperature in (D) is about 105° C. to 115° C., the condenser temperature in (D) is about 20° C. to 30° C., and the cold trap temperature in (D) is about −50° to −40° C.

15. The method of claim 10 wherein the distillation system includes an evaporator set to an evaporator temperature, a feed set to a feed temperature, a condenser set to a condenser temperature, and a cold trap set to a cold trap temperature, wherein the evaporator temperature in (E) is about 175° C. to 185° C., the feed temperature in (E) is about 105° C. to 115° C., the condenser temperature in (E) is about 105° C. to 115° C., and the cold trap temperature in (E) is about −50° to −40° C.

16. The method of claim 10 wherein the first concentration of terpenes in (D) is about 10% to 50%.

17. The method of claim 10 wherein the second concentration of terpenes in (E) is about 1% to 20%.

18. The method of claim 10 wherein the cannabinoid concentration of the decarboxylated crude obtained in (A) is about 50% to 85%.
